(19) United States
(12) Patent Application Publication
(10) Pub. No.: US 2025/0205119 A1
(43) Pub. Date: Jun. 26, 2025

(54) GRANULATE FOR A PERSONALIZED DENTAL DEVICE

(71) Applicant: ZINKH NV, Gent (BE)

(72) Inventor: Shane DE VREESE, Gent (BE)

(21) Appl. No.: 18/874,820

(22) PCT Filed: Jun. 13, 2023
(86) PCT No.: PCT/IB2023/056093

§ 371 Date: Dec. 13, 2024

(30) Foreign Application Priority Data
Jun. 13, 2022 (BE) 2022/5459

(51) Int. Cl. A61K 6/17; A61K 6/891; A61K 33/30

(52) U.S. Cl. CPC A61K 6/17; A61K 6/891; A61K 33/30

(57) ABSTRACT

The present invention relates to a granulate for manufacturing a personalized dental device, wherein the individual granulate of the granulate has a maximum particle size of 5 mm, wherein the granulate consist of a thermoplastic polymer composition, wherein the granulate forms a moldable paste at a temperature of at least 58° C. the polymer composition comprising at least 60% poly-ε-caprolactone (PCL) and at least a second polymer selected from styrene-based thermoplastics or polyolefin copolymers, and wherein the polymer composition comprises zinc ions and/or zinc salts at a concentration between 0.05% and 10%.

### TECHNICAL FIELD

The invention relates to a granulate for manufacturing a personalized dental device. The invention also relates to a method for manufacturing a personalized dental device by means of the granulate and a kit comprising the granulate.

### PRIOR ART

Mucogingival deformities and disorders are common findings in daily dental practice. Periodontal plastic surgery to address these functional and aesthetic requirements has become an integral part of periodontal treatments.

However, such surgical techniques are often associated with complications during and after surgery, such as excess blood loss and pain. Periodontal surgical procedures, for example, use autologous grafts, giving rise to pain and blood loss at the donor site. In addition, surgical stitching of the donor site often takes additional time.

Various dressing materials and covering methods are known from the literature.

For example, collagen and platelet-rich fibrin are commonly used postoperative dressing materials, for example to cover a postoperative wound, such as after tissue harvesting in the palate. However, it requires additional surgical time to secure the materials with sutures and there is also a risk of loss of material due to loosening of the sutures, friction from food or movement of the patient's tongue while eating and speaking. Early loss of material leads to a lot of postoperative pain and possible bleeding due to exposure of the rough surface of the wound. Palatal covering methods, such as retainers, are often uncomfortable to wear.

The present invention aims to find a solution to at least some of the above-mentioned problems, more specifically, the present invention aims, inter alia, to reduce operative and postoperative complications associated with periodontal plastic surgery procedures.

### SUMMARY OF THE INVENTION

The invention relates to a granulate for manufacturing a personalized dental device according to claim 1. More particularly, the invention relates to a granulate for manufacturing a personalized dental device, wherein the individual granulate of the granulate have a maximum particle size of 5 mm, wherein the granulate consist of a thermoplastic polymer composition, wherein the granulate forms a moldable paste at a temperature of at least 58° C., the polymer composition comprising at least 60% poly-ε-caprolactone (PCL) and at least a second polymer selected from styrene-based thermoplastics or polyolefin copolymers, and wherein the polymer composition comprises zinc ions and/or zinc salts at a concentration between 0.05% and 10% of the total weight.

This granulate makes it possible to form a personalized dental device that can be used during or after periodontal plastic surgery. The specific polymer composition makes it possible to easily form a moldable paste at a temperature of at least 58° C. Thanks to the possibility of personalization, the dental device fits nicely onto the desired location in the patient's mouth, allowing the wound to be adequately shielded while not hindering chewing and swallowing movements. In addition, the zinc ions present promote wound healing, reducing postoperative complications. In a preferred embodiment, the zinc ions and/or zinc salts are dispersed or dissolved in the polymer composition. The presence of a second polymer selected from styrene-based thermoplastics or polyolefin copolymers allows an optimal dispersion of these zinc ions and/or zinc salts. A good dispersion is necessary for optimal antimicrobial activity and consequently promotes wound healing.

In a further aspect, the invention relates to the use of the granulate according to claims 8-10. More particularly, the invention relates to the use of the granulate in wound covering and in the promotion of (postoperative) wound healing and (postoperative) pain treatment in the oral cavity of a patient.

In a further aspect, the invention relates to a method for manufacturing a personalized dental device according to claim 11. More in particular, the invention relates to a method for manufacturing a personalized dental device by means of the aforementioned granulate, comprising
a. Heating a dose of granulate to a temperature between 58 and 80° C. at which the granulate becomes a moldable paste;
b. forming the desired dental device from the paste and curing the molded device

In a final aspect, the invention relates to a kit for manufacturing a personalized dental device according to claim 14. More specifically, the invention relates to a method for manufacturing a personalized dental device comprising the aforementioned granulate and a workstation comprising one or more dosing receptacles and/or stirrers.

### DETAILED DESCRIPTION

The invention relates to a granulate for manufacturing a personalized dental device that reduces operative and postoperative complications after performing periodontal plastic surgery.

Unless otherwise defined, all terms used in the description of the invention, including technical and scientific terms, have the meanings commonly understood by those skilled in the art of the invention. For a better understanding of the description of the invention, the following terms are explained explicitly.

In this document, “a” and “the” refer to both the singular and the plural, unless the context presupposes otherwise. For example, “a segment” means one or more segments.

When the term “around” or “about” is used in this document with a measurable quantity, a parameter, a duration or moment, and the like, then variations are meant of approx. 20% or less, preferably approx. 10% or less, more preferably approx. 5% or less, even more preferably approx. 1% or less, and even more preferably approx. 0.1% or less than and of the quoted value, insofar as such variations are applicable in the described invention. However, it must be understood that the value of a quantity used where the term “about” or “around” is used, is itself specifically disclosed.

The terms “comprise”, “comprising”, “consist of”, “consisting of”, “provided with”, “have”, “having”, “include”, “including”, “contain”, “containing” are synonyms and are inclusive or open terms that indicate the presence of what follows, and which do not exclude or prevent the presence of other components, characteristics, elements, members, steps, as known from or disclosed in the prior art.

“Antimicrobial activity” as described in this document refers on the one hand to the polymer's inhibitory effect on microbial growth in the oral cavity and, on the other hand, to the antiseptic effect of the polymer, preventing microbial contamination of the dental device.

“Periodontal plastic surgery” as described in this document is defined as the collection of surgical treatments that are performed to prevent or correct developmental disorders and anatomical, traumatic, and pathological deviations of the gingiva, alveolar mucosa, and alveolar bone.

In the context of the present invention, the term “dental device” is understood to mean any device that can be placed in the oral cavity of a user, which will perform a protective, corrective, preventive and/or sterilizing function there.

Quoting numeric intervals by the endpoints includes all integers, fractions, and/or real numbers between the endpoints, including those endpoints.

Mucogingival deformities and disorders are common findings in daily dental practice. Periodontal plastic surgery to address these functional and aesthetic requirements has become an integral part of periodontal treatments. Various treatment methods, including free gingival grafts (FGG), connective tissue grafts (CTG), pedicle grafts, acellular dermal matrix (ADM) grafts, and guided tissue regeneration (GTR) are used to cover the exposed root surface and increase the width and thickness of the keratinized tissue.

Autologous connective tissue grafts can be harvested from the palate, maxillary tuberosity or edentulous areas; however the palate is the most common donor site.

In addition to connective tissue grafts, a bone graft is indicated in some cases. A bone graft is a surgical procedure to repair or rebuild bones by transplanting bone tissue. Autologous donor tissue can be used for this, for example a small block of bone from the back of the jaw.

The surgical wound at the donor site usually heals within 10 days and is often associated with postoperative discomfort for the patients due to the painful wound and the occurrence of bleeding, which can lead to eating and speaking disorders during the healing process. Also during the operation it is advantageous to stop the bleeding from the wound as efficiently and quickly as possible, in order to optimize the visibility during the operation.

The purpose of this invention is to provide a personalized dental device to alleviate morbidity during and after periodontal plastic surgery. For example, postoperative pain and bleeding are the two most common complications following tissue harvesting from a donor site. As described above, various dressing materials and covering methods are already known from the literature. However, these often require additional surgical time to secure the materials with sutures, present a risk of material loss or cause discomfort when wearing them.

In a first aspect, the invention relates to a granulate for manufacturing a personalized dental device, wherein the individual granulate of the granulate have a maximum particle size of 5 mm, wherein the granulate consist of a thermoplastic polymer composition, wherein the granulate forms a moldable paste at a temperature of at least 58° C., characterized in that the polymer composition comprises at least 60% poly-ε-caprolactone (PCL) and at least a second polymer selected from styrene-based thermoplastics or polyolefin polymers or copolymers, and wherein the polymer composition comprises zinc ions and/or zinc salts at a concentration between 0.05% and 10% of the total weight.

In an embodiment, the granulate forms a moldable paste at a temperature of at least 58° C. In an embodiment, the granulate forms a moldable paste at a temperature of at least 59° C. In an embodiment, the granulate forms a moldable paste at a temperature of at least 60° C. In an embodiment, the granulate forms a moldable paste at a temperature of at least 61° C. In an embodiment, the granulate forms a moldable paste at a temperature of at least 62° C. In an embodiment, the granulate forms a moldable paste at a temperature of at least 63° C. In an embodiment, the granulate forms a moldable paste at a temperature of at least 64° C. In an embodiment, the granulate forms a moldable paste at a temperature of at least 65° C. In an embodiment, the granulate forms a moldable paste at a temperature of at least 66° C. In an embodiment, the granulate forms a moldable paste at a temperature of at least 67° C. In an embodiment, the granulate forms a moldable paste at a temperature of at least 68° C. In an embodiment, the granulate forms a moldable paste at a temperature of at least 69° C. In an embodiment, the granulate forms a moldable paste at a temperature of at least 70° C. In an embodiment, the granulate forms a moldable paste at a temperature of at least 71° C. In an embodiment, the granulate forms a moldable paste at a temperature of at least 72° C. In an embodiment, the granulate forms a moldable paste at a temperature of at least 73° C. In an embodiment, the granulate forms a moldable paste at a temperature of at least 74° C. In an embodiment, the granulate forms a moldable paste at a temperature of at least 75° C. In an embodiment, the granulate forms a moldable paste at a temperature of at least 76° C. In an embodiment, the granulate forms a moldable paste at a temperature of at least 77° C. In an embodiment, the granulate forms a moldable paste at a temperature of at least 77° C. In an embodiment, the granulate forms a moldable paste at a temperature of at least 78° C. In an embodiment, the granulate forms a moldable paste at a temperature of at least 79° C. In an embodiment, the granulate forms a moldable paste at a temperature of at least 80° C.

The granulate according to the present invention comprises a thermoplastic polymer composition comprising at least 60% poly-ε-caprolactone (PCL). In an embodiment, the PCL has an average molecular weight between 10000 and 80000 g/mol, more preferably between 20000 and 70000 g/mol, more preferably between 40000 and 60000 g/mol, such as 50000 g/mol.

PCL is a semi-crystalline thermoplastic polymer with a low melting temperature, which makes it easy to process. PCL materials can be degraded in the environment by organisms such as fungi and bacteria. However, in the strict sense, they are not biodegradable in vivo: due to the lack of the necessary enzymes, PCL is not biologically degraded in the body of animals and humans. PCL is bioresorbable because the polymer chains are broken down through the relatively slow process of hydrolytic degradation.

The polymer composition comprises at least a second polymer selected from styrene-based thermoplastics or polyolefin polymers or copolymers. Addition of such a second polymer provides the polymer composition with the desired mechanical properties (such as the desired thermal expansion/shrinkage) and enables optimum dispersion of the zinc ions and/or zinc salts. Namely, the inventors have discovered that dispersion of the zinc component (the zinc ion and/or zinc salt) directly into PCL produces suboptimal results. As such, the second polymer ensures good compatibility between the various components of the polymer composition (particularly PCL, the zinc component, etc.). Making a masterbatch, where the zinc component is first dispersed in a carrier polymer (the second polymer), can help achieve better dispersion. This pre-mixing step ensures that the additive particles are evenly distributed before being incorporated into the PCL. The masterbatch, which contains a higher concentration of the zinc component, is then mixed with the PCL matrix in the correct proportions. This approach provides better dispersion of the zinc component in the PCL matrix and makes handling and uptake easier.

In an embodiment, the second polymer is a styrene-based thermoplastic, such as polystyrene-butadiene-styrene (SBS), polystyrene (PS), acrylonitrile-butadiene-styrene (ABS), acrylonitrile styrene acrylate (ASA), High Impact Polystyrene (HIPS), and styrene acrylonitrile (SAN). High Impact Polystyrene (HIPS) is a modified form of polystyrene with an elastomer, usually polybutadiene, to improve toughness and impact resistance. In a preferred embodiment, the second polymer is SBS. SBS can be mixed with PCL to improve its elastic properties. In a preferred embodiment, the second polymer is PS. In an embodiment, the polymer composition comprises 1-20% PS, preferably 1 to 15% PS, more preferably 1 to 10% PS. In an embodiment, the polymer composition comprises 1% PS. In an embodiment, the polymer composition comprises 2% PS. In an embodiment, the polymer composition comprises 3% PS. In an embodiment, the polymer composition comprises 4% PS. In an embodiment, the polymer composition comprises 5% PS. In an embodiment, the polymer composition comprises 6% PS. In an embodiment, the polymer composition comprises 7% PS. In an embodiment, the polymer composition comprises 8% PS. In an embodiment, the polymer composition comprises 9% PS. In an embodiment, the polymer composition comprises 10% PS.

In an embodiment, the second polymer is a polyolefin or a polyolefin copolymer, such as polyethylene (PE), polypropylene (PP), ethylene vinyl acetate (EVA), polyvinyl butyraldehyde (PVB), polybutylene (PB), polyisobutene (PIB), ethylene propylene diene monomer (EPDM). In a preferred embodiment, the second polymer is EVA. Addition of EVA as a second polymer ensures a reduced brittleness of the polymer composition. In an embodiment, the polymer composition comprises 0.1-40% EVA. In an embodiment, the polymer composition comprises 1-10% EVA, preferably 2-5% EVA, such as 3% EVA. In an embodiment, the polymer composition comprises 10-30% EVA, preferably 15-25% EVA, such as 20% EVA.

In an embodiment, the polymer composition comprises both a styrene-based thermoplastic and a polyolefin polymer or copolymer. In an embodiment, the polymer composition comprises PS and a polyolefin polymer or copolymer. In an embodiment, the polymer composition comprises 1-5% PS and 15-20% polyolefin polymer or copolymer. In an embodiment, the polymer composition comprises 3% PS and 17% polyolefin polymer or copolymer.

In the event that the PCL is present in a lower weight percentage than 60% in the granulate, a too low chemical binding force will be obtained. In addition, the melting point will rise, which reduces the processability for shape change. If the second polymer has a weight ratio that is too low, the granulate will not exhibit the desired mechanical properties (such as optimum thermal expansion) and/or an optimum dispersion of the zinc ions and/or zinc salts will not be obtained.

In an embodiment, the thermoplastic polymer composition furthermore comprises additional components to improve the mechanical properties (for example flow behavior, tensile strength, etc.) and/or the compatibility between the various components in the polymer composition. In an embodiment, the polymer composition furthermore comprises one or more additional components, such as one or more fillers, one or more plasticizers, one or more coloring agents and/or color stabilizers, one or more flavoring agents and/or one or more flow enhancing additives.

In an embodiment, the thermoplastic polymer composition comprises one or more thickeners, such as, for example, xanthan gum.

In an embodiment, the thermoplastic polymer composition further comprises one or more plasticizers as an additional component. In a preferred embodiment, plasticizers known from the food sector are added, such as, for example, xanthan gum or esters of fatty acids or sugars, such as, for example, sucrose esters. These additives increase the flexibility and reduce the brittleness of PCL. Plasticizers such as dioctyl phthalate (DOP) or polyethylene glycol (PEG) can be added to improve the toughness and elongation of PCL.

In an embodiment, the thermoplastic polymer composition further comprises one or more fillers as an additional component. In a preferred embodiment, these fillers comprise food-grade mineral fillers such as chalk, talc and/or clay. For example, such fillers can assist in curing the polymer composition, increasing the softening temperature, and mixing PCL with the second polymer. In an embodiment, the thermoplastic polymer composition comprises one or more flow enhancing additives such as lubricants to improve the flow properties of PCL during processing. Examples are stearates, glycerol monostearate or polyethylene wax.

In an embodiment, proteins and/or protein derivatives are added as an additional component to the polymer composition to increase melt strength. Since compounding preferably takes place at a relatively low temperature (preferably between 150° C. and 190° C.), it is possible to add proteins and/or protein derivatives without degrading them.

In an embodiment, the thermoplastic polymer composition comprises one or more flavoring agents and/or one or more coloring agents as an additional component. In an embodiment, the thermoplastic polymer composition comprises menthol as a flavoring agent. In an embodiment, the thermoplastic polymer composition comprises one or more color stabilizers, such as UV stabilizers and/or antioxidants. UV stabilizers protect PCL from degradation caused by UV radiation, preserving color stability. Examples are benzophenones, hindered amine light stabilizers (HALS) and UV absorbers. Antioxidants help prevent oxidation and discoloration of PCL. Commonly used antioxidants are sterically hindered phenols and phosphites.

By providing granulate with a maximum particle size of 5 mm and consisting of such a polymer composition as described above, the granulate can form a moldable paste at a temperature of at least 58° C. that is easy to personalize. As a result, the dental device connects nicely to the desired location in the oral cavity of the patient, so that the wound can be adequately shielded and at the same time chewing, speaking and swallowing movements are not hindered. In addition, the zinc ions present promote wound healing, reducing postoperative complications.

The zinc ions and/or zinc salts are contained in the polymer composition at a concentration between 0.05% and 10%, more preferably between 0.05% and 5%, more preferably between 0.05% and 3%, more preferably between 0.05% and 2%, more preferably between 0.05% and 1% of the total weight.

In addition to protecting surgical wounds, the granulate can also be used to fill up (partial) dentures, also described as relining. As a result, the dentures fit well on the jaw again and the bite height can also be restored. Relining of dentures is necessary because the toothless jaw always continues shrinking and therefore changes shape. Especially the first year after the extraction of the teeth, this change goes very quickly, only to slow down again later. The shrinking of the jaws will create space between the jaw and the dentures. As a result, the grip is less, but the pressure will also be less well distributed, so that shrinkage can go faster again. Relining can be performed directly in the patient's mouth or indirectly. In the indirect method, a precision impression of the jaw is taken with the dentures, after which a dental technician converts the impression material into synthetic resin. The indirect relining of the dentures can also be done with a border mold, so that the edges of the jaw ridge are accurately defined.

In an embodiment, the granulate has a melting temperature of between 58 and 80° C., preferably between 58 and 70° C., more preferably between 58 and 65° C., measured according to ISO 3146:2022. This melting temperature makes it possible to obtain a moldable paste when heated to an acceptable temperature, and to form a dental device within an acceptable time span of 30 seconds to 2 minutes, which retains its shape when cooled to room temperature.

The characterization of the mechanical properties of the polymer composition of the granulate according to the present invention was done according to the ISO 527-2:2012 standard.

In an embodiment, a film with a thickness of ±2 mm is made from the melt of the polymer composition. Samples of type 5A from the ISO 527-2:2012 standard are then punched from this film. Subsequently, the stress-strain curves of the polymer composition were measured at strain rates of 20 mm/min, 50 mm/min and 100 mm/min. Each experiment was repeated four times so that a total of twelve test samples were tested. All experiments were performed at 20° C.

In an embodiment, the yield stress Oy is between 2 and 50 MPa, preferably between 2 and 40 MPa, more preferably between 2 and 30 MPa, more preferably between 2 and 20 MPa, such as between 5 and 15 MPa. The modulus of elasticity E was determined according to the instructions of the ISO 527-2:2012 standard after converting the engineering stress o into the actual stress. In an embodiment, the global modulus of elasticity (i.e. without taking strain rate into account) is between 200 and 1500 MPa, more preferably between 200 and 1200 MPa, more preferably between 300 and 1000 MPa, more preferably between 400 and 700 MPa. In an embodiment, the tensile strength σT of the polymer composition is between 5 and 100 MPa, more preferably between 5 and 60 MPa, more preferably between 5 and 50 MPa, more preferably between 5 and 40 MPa, such as between 10 and 30 MPa.

In an embodiment, the elongation at break of the polymer composition of the granulate is between 500% and 1200%, more preferably between 600% and 900%, more preferably between 700% and 900%, such as 800%. The elongation at break is the deformation at which fracture occurs. Such a high value of the current polymer composition indicates a plastic material with a high elongation at break.

In an embodiment, the Melt Flow Index (MFI) of the polymer composition is between 5 and 15, more preferably between 5 and 10, such as 7, measured in accordance with NEN-EN-ISO 1133-1:2011.

In an embodiment, the water content of the polymer composition is extremely low (<1%), so that the dental device is unlikely to contain water and bacteria are less likely to remain, thereby maintaining hygiene.

In a preferred embodiment, the zinc ions and/or zinc salts are dispersed or dissolved in the polymer composition.

If the compatibility between the zinc ions and/or zinc salts and the polymer composition is insufficient, the polymer composition will release the metal ions over time. Therefore, in a preferred embodiment, the zinc ions and/or zinc salts are dispersed or dissolved in the polymer composition. This is achieved by adding the zinc ions and/or zinc salts to a solution or a dispersed phase of monomers before or during the polymerization reaction of these monomers. In this way, the zinc ions and/or zinc salts will be incorporated into the plastic composition and the compatibility between the plastic and the zinc ions and/or zinc salts will be such that the released concentration of zinc ion remains limited, even for extended periods. In an embodiment, an active mixture of zinc ions and/or zinc salts is mixed into a polymer composition by compounding. “Compounding” is the creation of formulations for the production of plastics and/or synthetic fibers by mixing/blending polymers and additives in a molten state. In a further preferred embodiment, the compounding takes place at a low temperature. The compounding preferably takes place at a temperature between 120 and 220° C., more preferably between 140 and 200° C., even more preferably between 150 and 190° C.

Also when the polymer composition is used in the manufacture of equipment, such as a dental device according to the present invention, the bond between the polymer and the metal salts will be such that the released concentration of zinc ions remains limited. This is important, especially for equipment that comes into close contact with the body, such as the oral cavity. This is because the release of too high a concentration of metal ions can lead to undesirable side effects. This can range from irritation to chronic intoxication and can even lead to serious conditions such as neurological problems. In an embodiment, the released concentration of the zinc ions when using the device in the oral cavity of a patient is up to 21 parts per million (ppm) more preferably between 0 and 5 ppm, more preferably between 0 and 1 ppm, more preferably between 0 and 0.5 ppm. Such exceptionally low to no release of metal ions can be described as “no leaching”.

In a preferred embodiment, no such values are exceeded when the device is exposed for at least 24 hours, preferably at least 36 hours, to an aqueous environment, such as for example the oral cavity.

Zinc ions and/or zinc salts are contained in the polymer composition at a concentration between 0.05% and 10% of the total weight.

In an embodiment zinc is present in the polymer composition in the form of a zinc salt, preferably zinc PCA, zinc oxide, zinc hydroxide, zinc pyrrolidone, zinc pyrithione, a zinc salt of a fatty acid or a mixture thereof.

Zinc is an essential trace element. Among other things, it plays an important role in energy production in the body, cell metabolism, DNA and RNA synthesis and regulation of the immune system. Furthermore, zinc is known for its antimicrobial properties. Zinc, for example, is present on the top layer of the skin and forms a defense barrier against viruses and bacteria. In recent years, numerous studies have confirmed the antibacterial and antiviral properties of zinc ions, especially against Gram⁺ bacteria such as streptococci and actinomycetes, important bacteria in the oral cavity. Indeed, zinc ions have bacteriostatic and bacteriocidal properties, which means that they can inhibit the growth of bacteria and block various biological processes that are fundamental for the survival of the bacteria themselves or for the formation of bacterial biofilms.

Although there is only very limited or no release of the metal ions into the oral cavity, the inventors of the present invention still found that the granulate has an antimicrobial activity. Without wishing to be bound by a theory, this could possibly be due to a positive influence of the zinc ions present on the functioning of the salivary glands, thereby influencing microbial growth and the formation of a charged ion field that prevents pathogens such as bacteria and viruses from adhering to the polymer and nearby structures. By stopping microbial growth in the presence of disease, the immune system has a chance to regain control of the infection and heal the body.

The inventors have discovered that dispersion of the zinc component (the zinc ion and/or zinc salt) directly into PCL produces suboptimal results. Experiments have shown that in order to obtain a good dispersion of the zinc component in the PCL-containing polymer composition, the zinc component must be pre-dispersed in a compatible second polymer before being added to the PCL polymer.

However, not every second polymer is suitable for obtaining a suitable dispersion of the zinc ion and/or zinc salt. This is because an optimal dispersion depends on the properties of the components, such as the flow behavior and the polarity. In addition, not every second polymer is compatible with PCL. The inventors have discovered that styrene-based thermoplastics or polyolefin polymers or copolymers are extremely suitable to function as a second polymer, as they are both suitable for optimally dispersing the zinc ion and/or zinc salt and are also compatible with the main component of the polymer composition, in particular PCL.

In a preferred embodiment, the zinc ions and/or zinc salts are therefore first dispersed or dissolved in the second polymer, before the mixture obtained is added to the PCL polymer in order to obtain the final polymer composition. In an embodiment, the zinc ions and/or zinc salts are first dispersed or dissolved in the second polymer at a concentration between 0.125% and 25%, preferably between 1% and 10%, more preferably between 1% and 5%, such as 1%, 2%, 3%, 4% or 5%.

In a preferred embodiment zinc is present in the polymer composition in the form of a zinc salt. In an embodiment, zinc is present in the form of zinc chloride (ZnCl₂), zinc sulfate (ZnSO₄), zinc acetate (Zn(CH₃COO)₂), zinc carbonate (ZnCO₃), zinc nitrate (Zn(NO₃)₂), zinc stearate, zinc PCA, zinc oxide, zinc hydroxide, zinc pyrrolidone, zinc pyrithione, a zinc salt of a fatty acid or a mixture thereof. In an embodiment, the zinc salt is present in a composition further comprising stabilizers, plasticizers, fatty acids and/or minerals.

The choice of which specific zinc ions and/or zinc salts are added to the polymer composition has an effect on the dispersion of these zinc components in the polymer composition. The inventors have discovered that not every zinc ion or zinc salt shows an equally optimal dispersion in the polymer composition (see Example 3). Zinc oxide (ZnO) is usually in the form of solid particles and may have a tendency to agglomerate or form clusters, especially at higher concentrations. This can result in poor dispersion within the PCL matrix. Achieving a uniform dispersion of ZnO in PCL may require additional process steps or techniques to break up the agglomerates and improve distribution.

On the other hand, zinc salts, such as zinc citrate, zinc stearate or zinc acetate, are often available in powder or granular form. These salts usually have smaller particle sizes and can show better dispersibility in PCL. In addition, zinc salts may have a higher affinity for PCL due to their similar polarities leading to better compatibility and dispersion.

In a preferred embodiment zinc is therefore not present in the polymer composition in the form of zinc oxide. The inventors have discovered that zinc oxide shows a suboptimal dispersion in the polymer composition and shows aggregation in the polymer composition after compounding (see Example 3). A good dispersion is necessary for an optimal antimicrobial activity of the granulate and therefore promotes wound healing.

In a preferred embodiment zinc is present in the polymer composition in the form of zinc citrate. Zinc citrate is obtained from the reaction between zinc oxide or zinc hydroxide and citric acid. The inventors have discovered that this form of zinc disperses optimally in the polymer composition and thus offers a granulate with an optimal antimicrobial activity. In an embodiment, the zinc citrate is present in a composition further comprising stabilizers, plasticizers, fatty acids and/or minerals.

In a preferred embodiment, the polymer composition comprises a zinc salt, the zinc salt being dispersed in the second polymer (selected from styrene-based thermoplastics or polyolefin polymers or copolymers), before the mixture obtained is added to the PCL polymer to obtain the final polymer composition.

In a further preferred embodiment, the polymer composition comprises a zinc salt, the zinc salt being dispersed in PS in a first step, before the mixture obtained is added to the PCL polymer to obtain the final polymer composition.

In an alternative preferred embodiment, the polymer composition comprises a zinc salt, the zinc salt being dispersed in EVA in a first step, before the mixture obtained is added to the PCL polymer to obtain the final polymer composition.

In a further preferred embodiment, the polymer composition comprises zinc citrate, the zinc citrate being dispersed in the second polymer (selected from styrene-based thermoplastics or polyolefin polymers or copolymers), before the mixture obtained is added to the PCL polymer to obtain the final polymer composition.

In a further preferred embodiment, the polymer composition comprises zinc citrate, the zinc citrate being dispersed in PS in a first step, before the mixture obtained is added to the PCL polymer to obtain the final polymer composition.

In an alternative preferred embodiment, the polymer composition comprises zinc citrate, the zinc citrate being dispersed in EVA in a first step, before the mixture obtained is added to the PCL polymer to obtain the final polymer composition.

In addition to a suitable compatibility between the various components, an optimal dispersion also depends on the conditions in which this step takes place, such as the temperature and the arrangement of the equipment (such as the screw speed, for example).

As described above, dispersion of the zinc component in the second polymer of the polymer composition takes place at a temperature between 120 and 220° C., more preferably between 140 and 200° C., even more preferably between 150 and 190° C.

As already mentioned, due to the concentration of zinc ions contained in the polymer composition, the dental device according to the present invention has an antimicrobial activity. As a result, the concentration of metal ions contained in the polymer composition also provides an antiseptic effect, limiting among other things bacterial growth and biofilm formation on the dental device. This offers advantages in the maintenance of the dental device, preventing bacterial contamination of the dental device. This minimizes bacterial proliferation in the oral cavity that could be caused by growth on the dental device.

In addition, when placed in the oral cavity, the device can effect a reduction in the amount of bacteria present there. This allows the device to protect an open wound through its antimicrobial activity, for example, and thus accelerate and ensure the healing of this open wound. Zinc is also known for its role in wound healing and promotes hemostasis, among other things. In addition, the specific granulate composition of the present invention, wherein the individual granulate of the granulate have a maximum particle size of 5 mm, wherein the granulate consist of a thermoplastic polymer composition, wherein the granulate forms a moldable paste at a temperature of at least 58° C. and wherein the polymer composition comprises at least 60% poly-ε-caprolactone (PCL) and at least a second polymer selected from styrene-based thermoplastics or polyolefin polymers or copolymers, makes it possible to make a personalized dental device (such as a wound dressing) that is suitable for optimally covering the postoperative periodontal area (e.g., a postoperative wound) and thus promote wound healing. For example, as described above, the dental device can be applied without prior stitching of the wound.

In an embodiment, the granulate is processed into a semi-finished product. Such a semi-finished product facilitates processing into the final product and may, for example, comprise a sheet of the polymer composition in pre-cut form with specific dimensions or an already (partially) preformed personalized dental device (for example a healing cap or a (partial) mouthguard).

In a further aspect, therefore, the invention relates to the use of the aforementioned granulate for operative or postoperative protection in the oral cavity, such as for wound covering. In an embodiment, the invention relates to the use of the aforementioned granulate in the promotion of (postoperative) wound healing in the oral cavity of a patient.

Operative wounds can occur, for example, when transplanting autologous tissue, which is necessary, for example, in the case of receding gums or a bone transplant.

Receding gums is the process in which the tissue surrounding the teeth pulls away from a tooth, exposing more of the tooth or tooth root. This can cause damage to the supporting bone. Receding gums are a common dental problem; 4% to 12% of adults suffer from it and it often goes unnoticed until it becomes more severe. To repair the damage and prevent further dental problems, a gum graft may be necessary.

Hard palate connective tissue grafting is a reliable grafting technique used for achieving root coverage and increasing the width and thickness of the keratinized tissue in periodontal plastic surgery. Morbidities at the donor site, including complications from postoperative bleeding, pain during the healing phase, difficulty eating and speaking, and unexpected healing patterns, are always a concern for physicians and patients alike. Autologous connective tissue grafts can be harvested from the palate, maxillary tuberosity or edentulous areas, with the palate being the most common donor site. However, the healing of the surgical wound at the donor site is often accompanied by postoperative discomfort for the patients due to the painful wound and bleeding, which can lead to eating and speaking disorders during the healing process.

In an embodiment, the invention relates to the use of the aforementioned granulate during or after a palatal graft operation.

A bone graft is a surgical procedure to repair or rebuild bones by transplanting bone tissue. Autologous tissue can be used for this, for example a small block of bone from the back of the jaw. Two-site intraoral surgery for the use of autogenous grafts has a significant increase in postoperative morbidity, and if an autogenous bone graft becomes exposed due to breakdown of the overlying tissues, graft loss may result.

In an embodiment, the invention relates to the use of the aforementioned granulate during or after a bone graft operation.

The extraction of a tooth triggers a cascade of events that result in resorption of the alveolar bone surrounding the tooth socket. The buccal bone loss that occurs after extraction leads to vertical and horizontal bone loss. Complex hard and soft tissue reconstruction is required to achieve aesthetically acceptable results in such cases. In the socket-shield technique (SST), the root is cut and the buccal two-thirds of the root is retained in the socket, leaving the periodontium intact along with the bundle bone and buccal bone.

In an embodiment, the invention relates to the use of the aforementioned granulate during or after a socket-shield operation.

By adequately covering the surgical wound, this wound can heal better. The granulate can, as it were, be modeled into a “band-aid” that accurately covers the wound, without the need for stitching the wound or attaching the “band-aid”. Due to the moldable nature of the granulate, the band-aid can be personalized based on the specific needs of the patient. The zinc ions present also promote wound healing.

The dental device can also be used during the operation to cover the wound and thus stop the bleeding or stabilize the blood clot. This increases visibility during the operation.

In a further aspect, the invention relates to the use of the aforementioned granulate for use in (postoperative) pain treatment in the oral cavity of a patient.

As discussed above, such a periodontal surgical procedure (such as a graft) is often associated with postoperative pain. However, by personalizing the covering of the wound and the dental device, pain can be minimized. The personalization ensures that the device can be placed in the desired place in the oral cavity and remains anchored there. As a result, the patient will not be bothered by a detaching graft, and the patient will also be safeguarded from speaking, swallowing and chewing problems associated with a sub-optimally placed dental device. In an embodiment, pain is measured by a VAS scale, also referred to as a VAS score or visual analog scale. The VAS scale works as follows. The patient is asked if they are in pain. If the patient answers yes, they are asked to indicate the pain on a 10 centimeter horizontal line. On the left is no pain, on the right is worst imaginable pain. A line is placed at the designated spot. The score is determined based on how many centimeters from the left the line is placed. In this scale we have three categories. Pain score one through three is considered mild pain. Scores four through seven are considered moderate pain. Any score above seven is considered severe pain. The scores given are called the VAS score. In an embodiment, the VAS score is determined immediately after surgery. In an embodiment, the VAS score is determined one week after surgery. In an embodiment, the VAS score is determined two weeks after surgery. In an embodiment, the VAS score is determined three weeks after surgery. In an embodiment, the VAS score is determined 4 weeks after surgery. In an embodiment, the average VAS score of a patient using the granulate of the present invention is at least 10% lower, preferably at least 20% lower, preferably at least 30% lower than said patient's average VAS score when not using the granulate according to the present invention.

In a further aspect, the invention relates to a method for manufacturing a personalized dental device by means of the aforementioned granulate, comprising:
a. Heating a dose of granulate to a temperature between 58 and 80° C. at which the granulate becomes a moldable paste;
b. forming the desired dental device from the paste and curing the molded device

Such a method is easy to perform by an attending physician or surgeon in the operating room. The heating of a dose of granulate can be done with any suitable heating technique known in the art. In an embodiment, a dose of granulate is mixed with a heated liquid, the heated liquid having a temperature between 58° C. and 80° C., wherein the granule becomes a moldable paste that is then taken out of the heated liquid and formed and cured into the dental device within a specified time period. In a further embodiment, the dose of granulate is mixed with a heated liquid by means of a stirrer. In a preferred embodiment, the dose of granulate is mixed with a heated liquid by means of a stirrer for less than 30 seconds, preferably less than 20 seconds.

In a preferred embodiment, the shaping and curing of the formed device takes place within a period of 5 minutes, preferably within a period of 4 minutes, more preferably within a period of 3 minutes, more preferably within a period of 2 minutes.

In an embodiment, a dose of granulate is between 1 and 20 grams, depending on the desired application. In an embodiment, the dose of granulate is between 5 and 15 grams, such as 10 grams for use in postoperative wound dressing in palatal graft surgery. In an embodiment, the dose of granulate is between 0.5 and 5 grams, such as 2 grams for use in postoperative wound dressing in bone grafting surgery.

In an embodiment, the desired dental device is formed by means of a piston. In an embodiment, when the dose of granulate is mixed with a heated liquid, the piston can be used to form and remove the moldable paste from the heated liquid. As described above, in an embodiment of the method a semi-finished product is formed which can subsequently be further processed into a personalized dental device.

In a further aspect, the invention relates to a kit for manufacturing a dental device, comprising the aforementioned granulate and a workstation comprising one or more dosing receptacles and/or stirrers.

In an embodiment, a volume of 10 grams of granulate is packed in a receptacle. In a preferred embodiment, the kit comprises multiple doses of granulate.

In a preferred embodiment, the kit further comprises a piston for forming the granulate into the dental device

In what follows, the invention is described by way of non-limiting examples illustrating the invention, and which are not intended to and should not be interpreted as limiting the scope of the invention.

### EXAMPLES

### Example 1

The granulate of the present invention is used for manufacturing a dental device, more specifically for manufacturing a wound dressing for covering a wound after a palatal graft. The individual granulate of the granulate have a maximum particle size of 5 mm and consist of a thermoplastic polymer composition comprising 97% poly-ε-caprolactone (PCL) and 3% polystyrene (PS). This results in a polymer composition with optimum mechanical properties (such as a sufficiently high chemical binding force, an optimum melting point and an optimum thermal expansion), making it possible to obtain a moldable paste at a temperature of at least 58° C. Zinc ions and/or zinc salts are contained in the polymer composition at a concentration of about 0.1% by weight. Due to the concentration of zinc ions contained in the polymer composition, the wound dressing according to the present invention has an antimicrobial activity. As a result, the concentration of metal ions contained in the polymer composition also provides an antiseptic effect, limiting among other things bacterial growth and biofilm formation on the dental device. This offers advantages in the maintenance of the dental device, preventing bacterial contamination of the dental device. This minimizes bacterial proliferation in the oral cavity that could be caused by growth on the dental device.

In addition, when placed in the oral cavity, the device can effect a reduction in the amount of bacteria present there. This allows the device to protect an open wound through its antimicrobial activity and thus accelerate and ensure the healing of this open wound. Zinc is also known for its role in wound healing and promotes hemostasis, among other things. The dental device is manufactured by heating up a dose of granulate. For this, water is heated to about 100° C. in a glass and cooled to about 75° C. At this temperature, 10 grams of the granulate of the present invention are added to the glass and the whole is stirred for 10 to 15 seconds until the granulate become flexible and form a moldable paste. Subsequently, the moldable paste is taken out of the heated liquid and formed into a disk of the desired size. This disk is placed in the mouth to cover the surgical wound there. Due to the moldable nature of the granulate, the dental device can be personalized based on the specific needs of the patient. After 30 to 60 seconds, the moldable paste starts to harden. After two minutes, the moldable paste is completely cured.

### Example 2

Twenty patients diagnosed with mucogingival deformities or insufficiently keratinized tissue around dental implants were included in the study of the present example. After grafting of connective tissue from the hard palate, the donor site was shielded with a collagen wound dressing (10 patients, Group 1) or with a wound dressing manufactured using the granulate of the present invention as discussed in Example 1 (also 10 patients, Group 2).

The dental device manufactured using the granulate of the present invention (Group 2) was applied to the donor site without any other dressing material or suture immediately after the harvesting procedure to stabilize the bleeding.

Patients were instructed to wear the dressing for the first 48 hours without removing it. From 48 hours to 1 week after surgery, patients were allowed to gently remove the dressing for cleaning. In the second week, the dressings were placed only during meals to prevent the friction of food on the palate while eating.

The patients returned to their normal lives and no longer wore the device from two weeks after surgery. Pain score of the donor site and discomfort from wearing the dressing were recorded 1 week, 2 weeks and 1 month after surgery. The Visual Analog Scale (VAS) was used to rate postoperative pain, with “no pain” as 0 to “worst pain” as 10 in the score. Bleeding in the postoperative healing phase and discomfort of wearing the dressing were evaluated with yes/no verbal questions, including the influence on phonetics, chewing and swallowing. Bleeding tendency was evaluated at the end of the procedure.

Since the dental device manufactured using the granulate of the present invention can be personalized, the patients in Group 2 experienced minimal postoperative pain and discomfort (the VAS score varied between 0 and 2 at all time points). Both chewing and swallowing were also not affected while wearing the dental device made from the granulate of the present invention. Bleeding at the donor site could also be minimized quickly and easily. In contrast, the patients in Group 1 experienced more discomfort from wearing the collagen dressing and reported a mean VAS score that was at least 10% higher than those in Group 2 at all time points.

### Example 3

Zinc provides antibacterial properties to the granulate and thus to the personalized dental device. However, it is important here that the zinc component (being the zinc ion and/or zinc salt) is sufficiently well dispersed in the polymer composition. Different compositions were tested to verify the dispersion of the zinc component in the polymer composition of the granulate. Various zinc components were also tested, in particular zinc oxide and zinc citrate.

Initially, the dispersion of both zinc components in PCL with a molecular weight of 50000 g/mol was tested. For this, both zinc oxide and zinc citrate were separately mixed directly into PCL, both at a concentration of 0.1% relative to the total weight of the polymer composition. Poor dispersion in PCL was found for both zinc components. This poor dispersion was optically visible, as the zinc components showed granulation and a suboptimal dispersion in the PCL polymer.

To solve this problem, it was decided to first mix the zinc component into a second polymer at a concentration of 3% before adding the resulting composition to PCL.

Mixing in of the zinc component was tested in several second polymers, namely polystyrene (PS), ethylene vinyl acetate (EVA), polymethyl methacrylate (PMMA) resin and polyethylene terephthalate (PET). Then the resulting composition was compounded with the PCL polymer, adding 3% of the resulting composition (comprising zinc component and second polymer) to the 97% of the PCL polymer. Mixing in of zinc oxide (ZnO) produced poor dispersion (granulation) in each of the second polymers tested and in the resulting polymer composition comprising PCL. This granulation was visible, among other things, when the polymer composition was melted again: some parts of the polymer composition did not melt at the predetermined melting temperature of ±60° C. This shows that ZnO has a greater tendency to form grains than zinc citrate. Moreover, only the mixing in of zinc citrate in EVA and PS resulted in good dispersion. This was not the case when zinc citrate was mixed in PMMA resin, nor in PET.

The antimicrobial activity of the different polymer compositions was moreover determined according to ISO 22196:2011. Only the polymer compositions comprising zinc citrate in combination with either PS or EVA as a second polymer showed a reduction in bacterial growth of more than 90%.

These experiments show that in order to obtain a good dispersion of the zinc component in the PCL-containing polymer composition, the zinc component must be pre-dispersed in a compatible second polymer before being added to the PCL polymer. Moreover, not every second polymer is suitable for obtaining a suitable dispersion of the zinc ion and/or zinc salt and not every zinc ion and/or zinc salt is suitable either. This is because an optimal dispersion depends on the properties of the components, such as the flow behavior and the polarity. In addition, not every second polymer is compatible with PCL. The inventors have discovered that zinc citrate is a suitable zinc salt for dispersion (unlike zinc oxide) and that PS and EVA are extremely suitable to function as a second polymer, as they are both suitable for optimally dispersing the zinc citrate and are also compatible with the main component of the polymer composition, in particular PCL.

1. A granulate for manufacturing a personalized dental device, wherein an individual granulate of the granulate has a maximum particle size of 5 mm, wherein the granulate consist of a thermoplastic polymer composition, wherein the granulate forms a moldable paste at a temperature of at least 58° C., characterized in that the thermoplastic polymer composition comprises at least 60% poly-ε-caprolactone (PCL) and at least a second polymer selected from styrene-based thermoplastics or polyolefin polymers or copolymers, and wherein the thermoplastic polymer composition comprises zinc ions and/or zinc salts at a concentration between 0.05% and 10% of the total weight.

2. The granulate according to claim 1, wherein zinc is present in the thermoplastic polymer composition in the form of a zinc salt.

3. The granulate according to claim 1, wherein the granulate has a melting temperature of between 58 and 70° C. measured in accordance with EN-ISO 1133-1:2011.

4. The granulate according to claim 1, wherein the second polymer is polystyrene (PS).

5. The granulate according to claim 1, wherein the second polymer is ethylene vinyl acetate (EVA).

6. The granulate according to claim 1, characterized in that the zinc ions and/or the zinc salts are dispersed or dissolved in the thermoplastic polymer composition.

7. The granulate according to claim 1, wherein the thermoplastic polymer composition furthermore comprises one or more fillers, one or more plasticizers, one or more coloring agents and/or color stabilizers, one or more flavoring agents and/or one or more flow enhancing additives.

8. A method of dressing a wound in an oral cavity of a patient, the method comprising administering to the patient the granulate according to claim 1.

9. A method of promoting postoperative wound healing in an oral cavity of a patient, the method comprising administering to the patient the granulate according to claim 1.

10. A method of treating postoperative pain in an oral cavity of a patient, the method comprising administering to the patient the granulate according to claim 1.

11. A method for manufacturing a personalized dental device by a granulate according to claim 1, comprising:
heating a dose of the granulate to a temperature between 58° C. and 80° C. at which the granulate becomes a moldable paste;
forming the personalized dental device from the moldable paste; and
curing the personalized dental device.

12. The method according to claim 11, wherein the personalized dental device is formed by a piston.

13. The method according to claim 11, wherein a semi-finished product is formed.

14. A kit for manufacturing a personalized dental device, comprising the granulate according to claim 1, and a workstation comprising one or more dosing receptacles and/or stirrers.

15. A kit according to claim 14, further comprising a piston for forming the granulate into the personalized dental device.
